(19) United States
(12) Patent Application Publication
(10) Pub. No.: US 2022/0096855 A1
(43) Pub. Date: Mar. 31, 2022

(54) METHOD AND APPARATUS FOR REALIZING AIR CHARGED PARTICLE WAVE WITH REQUIRED FREQUENCY

(71) Applicant: Yanbing LIU, Jinan City, Shandong (CN)

(72) Inventors: Yanbing LIU, Shandong (CN); Qijia LIU, BC, CA (US); Qirui LIU, BC, CA (US)

(21) Appl. No.: 17/427,083

(22) PCT Filed: Apr. 18, 2019
(86) PCT No.: PCT/CN2019/083285

§ 371 Date: Jul. 29, 2021

(30) Foreign Application Priority Data
Feb. 2, 2019 (CN) 201910106917.2

(51) Int. Cl. A61N 1/44; H01J 37/24

(52) U.S. Cl. CPC A61N 1/44; H01J 37/24

(57) ABSTRACT

The present disclosure relates to a method and apparatus for realizing an air charged particle wave with a required frequency. The method comprises the following step: controlling, by a waveform control unit, a charged particle generation unit, so that the charged particle generation unit generates a charged particle wave at a preset frequency. The apparatus comprises a charge and charged particle generation unit, wherein the unit is used for generating positively-charged particles or negatively-charged particle in the air; and a charged particle waveform control unit, wherein the waveform control unit is used for regulating the generation by the charged particle generation unit to control the density of the charged particles transmitted in the air, so that the particles in the air form a particle wave with a controllable density and movement direction in a predetermined waveform.

### TECHNICAL FIELD

The present disclosure relates to a method and apparatus for realizing an air charged particle wave with a required frequency.

### BACKGROUND

Charged particles refer to charged microparticles in various forms, and can be positively-charged positive ions or negatively-charged negative ions and charged spatial inherent particles and the like. Collision or contact of the charged particles with a certain energy, a certain conversion frequency and movement direction is conducive to health and physical therapy and healthcare.

Negative air ions are widely applied charged particles and are usually generated by ionizing air by means of a high voltage or a strong ray, which are conducive to physical and psychological health of the human body and primarily improve the function of the human by means of a nervous system and blood circulation of a human to promote health. The negative air ions can make a cerebral cortex inhibiting process of a human strengthen and adjust the function of the cerebral cortex, such that the negative air ions can play roles of calming, hypnotizing and reducing blood pressure.

In an existing charged particle generation system or apparatus, the charged particles are emitted randomly. Only the amount of the charged particles is emphasized without intentionality and directionality. The movement direction, density, frequency and direction cannot be regulated and controlled, and moreover, the charged particles are relatively low in energy and poor in physical therapy effect.

### SUMMARY

The embodiments of the present disclosure provide a method and apparatus for realizing an air charged particle wave with a required frequency. The method and structure are reasonable in design. Charged particles propagate in the air in a form of a charged particle wave matched with various physiological waveforms of a human body, and the charged particles with the required frequency can be regulated and output under different application scenes, the obtained charged particle wave with different frequencies and charge amounts is regulated and controlled physiologically correspondingly to meet an application requirement of treating and improving health. The method and apparatus are wide in application range, and solve problems existing in the prior art.

The technical solution adopted by the present disclosure to solve the technical problems is as follows:

a method for realizing an air charged particle wave with a required frequency, including the following step:

controlling, by a waveform control unit, a charged particle generation unit, so that the charged particle generation unit generates a particle wave at a preset frequency.

Controlling, by the waveform control unit, the charged particle generation unit includes the following steps:

reading, by the charged particle generation unit, a charged particle waveform pre-stored in the charged particle generation unit according to a user requirement;

carrying out amplification treatment on the charged particle waveform;

converting the charged particle waveform; and

generating the charged particle wave according to the charged particle waveform and transmitting the generated charged particle wave to the waveform control unit to enable the waveform control unit to regulate the charged particle wave.

Controlling, by the waveform control unit, the charged particle generation unit includes the following step:

transmitting, by the waveform control unit, particles to the charged particle generation unit according to the preset frequency to enable the charged particle generation unit to process the particles so as to form the charged particle wave with a specific frequency.

An apparatus for realizing an air charged particle wave with a required frequency, the apparatus including:

a charged particle generation unit, wherein the charged particle generation unit is configured to generate charged particles; and

a waveform control unit, wherein the waveform control unit is configured to change the charge energy of the charged particles generated by the charged particle generation unit, such that the particles form a particle wave in a predetermined waveform.

The charged particle generation unit includes:

a storage module configured to pre-store a set charged particle wave by the charged particle generation unit,

an amplification module configured to amplify the charged particle waveform by the charged particle generation unit,

a digital-to-analogue conversion module configured to convert the charged particle waveform by the charged particle generation unit, and

an execution module configured to generate charged particles by the charged particle generation unit according to the particle waveform.

The waveform control unit is a fan, configured to change an output amount of the charged particles by regulating an air speed of the fan.

The waveform control unit is an air channel, and the air channel is provided with a closing mechanism, configured to change an output amount of the charged particles by regulating a dimension of the air channel.

By adopting the above structure, by matching the charged particle generation unit with the waveform control unit, different charged particle wave output frequencies are modulated to be suitable for different application scenes. In a real life, brain waves acting on the human body are mainly divided into α waves, β waves, θ waves, δ waves and Schumann waves, wherein an oscillation frequency of the α waves is 8-13 Hz, an oscillation frequency of the β waves is 13-14 Hz, an oscillation frequency of the θ waves is 4-7 Hz, an oscillation frequency of the δ waves is 0-4 Hz, and an oscillation frequency of the Schumann waves is 7.83 Hz. The brain waves correspond to different function states of the human body. The apparatus outputs the charged particle waves in different waveforms to assist a user according to the function state of the user body. The charged particle waves with different frequencies are obtained according to the output waveforms in the control unit by means of the execution module, and the execution module can be a power supply, a fan or an air channel. By amplifying the output waveforms pre-stored in the control unit by regulating an amplifier chip, the output waveforms are more precise, such that the apparatus has the advantages of large application range and high practicality.

### BRIEF DESCRIPTION OF THE DRAWINGS

FIG. 1 is a flow schematic diagram of Embodiment 1 of the present disclosure.

FIG. 2 is a flow schematic diagram of Embodiment 2 of the present disclosure.

FIG. 3 is an electrical schematic diagram of the present disclosure.

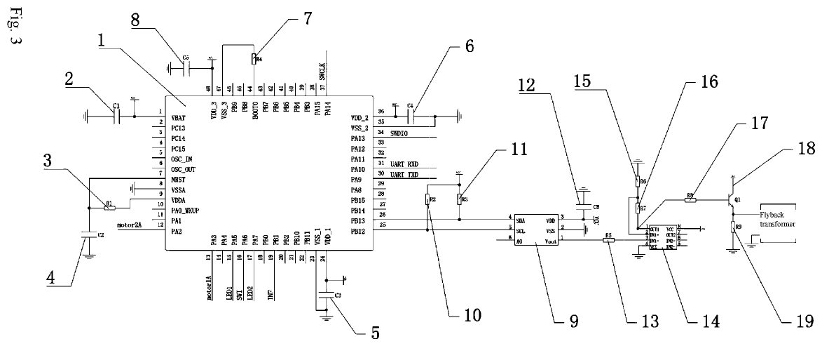

In the drawings, 1, microcontroller, 2, first capacitor, 3, first resistor, 4, second capacitor, 5, third capacitor, 6, fourth capacitor, 7, fourth resistor, 8, fifth capacitor, 9, digital-to-analogue conversion chip, 10, second resistor, 11, third resistor, 12, sixth capacitor, 13, fifth resistor, 14, amplifier chip, 15, sixth resistor, 16, seventh resistor, 17, eighth resistor, 18, triode, and 19, ninth resistor.

### DETAILED DESCRIPTION

In order to describe the technical features of the solution clearly, detailed description will be made to the present disclosure below by means of specific embodiments in combination with drawings thereof.

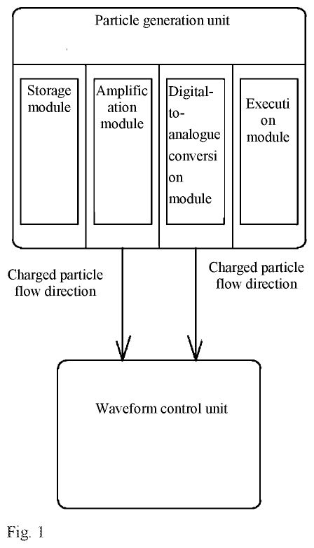

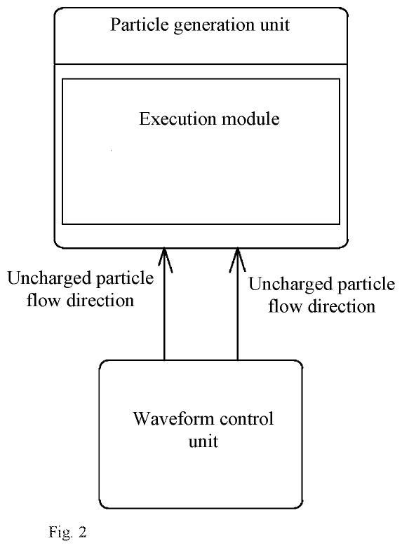

As shown in the FIGS. 1-2, an apparatus for realizing an air charged particle wave with a required frequency, the apparatus including:

a charged particle generation unit, wherein the charged particle generation unit is configured to generate charged particles;

a waveform control unit, wherein the waveform control unit is configured to change the density, frequency, direction and energy of the charged particles generated by the charged particle generation unit, such that the particles form a charged particle wave in a predetermined waveform.

The charged particle generation unit includes:

a storage module configured to pre-store a set charged particle wave by the charged particle generation unit,

an amplification module configured to amplify the charged particle waveform by the charged particle generation unit,

a digital-to-analogue conversion module configured to convert the charged particle waveform by the charged particle generation unit, and

an execution module configured to generate charged particles by the charged particle generation unit according to the charged particle waveform.

The waveform control unit is a fan, configured to change an output amount of the charged particles by regulating an air speed of the fan.

The waveform control unit is an air channel, and the air channel is provided with a closing mechanism, configured to change an output amount of the charged particles by regulating a dimension of the air channel.

a method for realizing an air charged particle wave with a required frequency, including the following step:

controlling, by a waveform control unit, a charged particle generation unit, so that the charged particle generation unit generates a charged particle wave at a preset frequency.

Controlling, by the waveform control unit, the charged particle generation unit includes the following steps:

reading, by the charged particle generation unit, a charged particle waveform pre-stored in the charged particle generation unit according to a user requirement;

carrying out amplification treatment on the charged particle waveform; converting the charged particle waveform;

generating the charged particle wave according to the charged particle waveform and transmitting the generated charged particle wave to the waveform control unit to enable the waveform control unit to regulate the charged particle wave.

Controlling, by the waveform control unit, the charged particle generation unit includes the following step:

transmitting, by the waveform control unit, particles to the charged particle generation unit according to the preset frequency to enable the charged particle generation unit to process the charged particles so as to form the charged particle wave with a specific frequency.

In an actual embodiment, an apparatus for realizing an air charged particle wave with a required frequency includes a microcontroller, an executor and a charged particle generator. The microcontroller is connected with the executor, and the executor is connected with the charged particle generator; a model number of the microcontroller is an STM32 serial chip, the microcontroller is provided with 48 pins, a first pin is connected with a first capacitor, and the first capacitor is earthed; a seventh pin is connected with a first resistor, the first resistor is connected with a ninth pin, the seventh pin is connected with a second capacitor, and the second capacitor is earthed; a twenty-fourth pin is connected with a third capacitor, the third capacitor is connected with a twenty-third pin and is earthed respectively; a thirty-sixth pin is connected with a fourth capacitor, and the fourth capacitor is connected with a thirty-fifth pin and is earthed, respectively; a forty-fourth pin is connected with a fourth resistor, and the fourth resistor is connected with a forty-seventh pin; a forty-eighth pin is connected with a fifth capacitor, and the fifth capacitor is earthed; the first pin, the twenty-fourth pin, the thirty-sixth pin and the forty-eighth pin are connected with a power supply end respectively; a twelfth pin and a thirteen pin of the microcontroller are fan pins, a fifteen pin and a seventh pin of the microcontroller are LED indicator light pins, a sixteenth pin of the microcontroller is a control button pin, a nineteenth pin of the microcontroller is a remote controller pin, a thirtieth pin and a thirty-first pin of the microcontroller are serial port pins, a thirty-fourth pin and a thirty-seventh pin of the microcontroller are program burning pins, and the microcontroller is connected with a memory through the thirty-fourth pin and the thirty-seventh pin, such that the memory transmits the pre-stored charged particle waveform to the microcontroller.

The microcontroller is further connected with a digital-to-analogue conversion chip, the digital-to-analogue conversion chip is provided with six pins, the microcontroller is connected with a fourth pin and a fifth pin on the digital-to-analogue conversion chip via a twenty-fifth pin and a twenty-sixth pin, the twenty-fifth pin of the microcontroller is connected with a second resistor, the second resistor is connected with a third resistor, the third resistor is connected with the twenty-sixth pin, and the twenty-fifth pin and the twenty-sixth pin are connected with the power supply end respectively.

The model number of the digital-to-analogue conversion chip is one of DAC5571, DAC5573 or DAC5574. A second pin of the digital-to-analogue conversion chip is earthed, a third pin of the digital-to-analogue conversion chip is connected with a sixth capacitor, the sixth capacitor is earthed, and the third pin is further connected with the power supply end; and a first pin of the digital-to-analogue conversion chip is connected with a fifth resistor, and the fifth resistor is connected with an amplifier chip.

The model number of the amplifier chip is LM386, the amplifier chip is provided with eight pins, an eighth pin is connected with a power supply, and a fourth pin is earthed; a third pin is connected with the fifth resistor, and the fifth resistor is connected with the first pin of the digital-to-analogue conversion chip; a second pin is connected with a sixth resistor, the sixth resistor is earthed, the second pin is connected with a seventh resistor and the seventh resistor is connected with a first pin; the first pin is connected with an eight resistor, the eighth resistor is connected with a triode, the triode is connected with the particle generator, and the particle generator is earthed; the triode is a NPN type triode, the first pin of the amplifier chip is connected with the eighth resistor, the eighth resistor is connected with a base electrode of the triode, a collecting electrode of the triode is connected with the power supply, an emitting electrode of the triode is connected with the particle generator, the emitting electrode of the triode is connected with a ninth resistor, and the ninth resistor is earthed.

The charged particle generator is a flyback transformer, and the model number of the flyback transformer is SKR1W12VDC1. The executor includes a voltage source, a fan or an air channel. The executor is further provided with a directional circulation dredging apparatus. The output waveform of the charged particle wave can also be adjusted by turning on and off the directional circulation dredging apparatus, thereby achieving an effect of controlling the charged particle wave.

A voltage value of the power supply end is 3.3 V and a voltage value of the power supply is 15 V.

During use, the microprocessor is connected with the executor, and a control program in the memory is input into the microcontroller 1 via the program burning pins on a microcontroller 1, such that the microcontroller 1 acts to be matched with a peripheral circuit on the microcontroller 1, and different particle wave output waveforms can be output in different application scenes according to a requirement of a user. Compared with an existing charged particle preparation apparatus, the apparatus of the present disclosure has the advantages that the charged particles are output in the form of pulse and charged particle waves, such that it can be convenient to adjust correspondingly according to the requirement of the user, and therefore, the apparatus can be suitable for various different environments and occasions; four bran waves acting on the human body will appear in different function states of the human body, for example, the α brain wave appears in initial sleep or initial awakening, and at the moment, the body function of the user can be in a relaxed state and has conscious awareness; the β wave will appear when the user is awake, and at the moment, the body function of the user is in a working state and attention is focused; the δ wave and the θ wave will appear when the user is asleep, and at the moment, the body function of the user is in a rest state. According to the principle, the apparatus can output particle waves of different waveforms to assist the user according to the body state of the user, such that the user can acquire higher-quality sleep and a more efficient working state; in many frequencies, there is still a Schumann wave with the frequency of 7.83 Hz, which is conducive to the mind and body of the user, and the apparatus can also emit the particle waves with this frequency, which is equivalent to ‘charge’ the body of the user. In a word, the apparatus can provide beneficial particle waves to the body of the user, such that the user is in a good working state or rest state; the waveform control unit can be one of the fan and the air channel. When the executor is the fan, the microcontroller regulates the air speed of the fan according to a specific frequency, such that the output particle waves form the specific waveforms; when the executor is the air channel, the microcontroller changes the output amount of the particles by regulating the dimension of the air channel, so as to form the specific waveform of the particle wave; the directional circulation dredging apparatus connected to the executor can transmit charges of the human body to the ground, such that the physical therapy effect of the charged particle wave is improved. The charged particle flow directions of the charged particle generation unit and the waveform control unit have two different modes. One of the modes is as follows: the charged particle generation unit processes the particles according to the specific frequency, such that the particles are charged, and the waveform control unit inspects the charged particles to form the charged particle wave needed by the user; the other mode is as follows: the waveform control unit regulates the content of the particles input into the charged particle generation unit, such that the particles input into the charged particle generation unit are processed according to the specific waveform to charge the particles and form the charged particle wave; the microcontroller 1 is further connected with the digital-to-analogue conversion chip 9 and the amplifier chip 14, such that a signal transmitted to the flyback transformer is more precise, the action error of the flyback transformer is reduced, and therefore, the charged particle waveform is free of a distorting phenomenon. In the apparatus, a waveform generation module can be arranged at one side of a power supply module or can be arranged in a high voltage generation module, which can be selected voluntarily according to the requirement of the user; the fan pin on the microcontroller 1 can be connected with a function assembly such as a heat dissipating fan to dissipate heat of each module in the apparatus. The button pin and the remote controller pin make the user's operation more convenient, the serial port pins can be connected with a terminal apparatus, and the user can detect the microcontroller in real time to prevent malfunction and chaos phenomena of the apparatus. The model number of the microcontroller 1 is the STM32 serial chip which is low in cost and good in universality. The model number of the flyback transformer is SKR1W12VDC1, the flyback transformer is excellent in performance and can be suitable for various environments and occasions. Under a common condition, the charged particles are all charged negative ions, among which negative air ions are widely applied, which facilitates the physical and psychological health of the human body. The negative air ions are output outward in a form of particle wave, and the apparatus is convenient to adjust, large in application range and high in practicality. The charged particle waves with different frequencies obtained by changing the output waveforms by the microcontroller 1 are applied to different environments, such that the control unit emits the charged particles with the specific waveforms (frequencies). The directional circulation dredging apparatus is additionally provided with a control module, which can achieve the effect of controlling the waveform of the charged particles. In a word, the apparatus of the present disclosure is wide in application field and can be applied to the fields of medicine, military project, environmental protection and the like.

The specific embodiments cannot be used as limitations to the protection scope of the present disclosure. For those skilled in the art, any replacements, improvements or transformations made on the embodiments of the present disclosure shall fall within the protection scope of the present disclosure.

What are not described in detail in the present disclosure are well-known technologies of those skilled in the art.

1. A method for realizing an air charged particle wave with a required frequency, comprising the following step:
controlling, by a waveform control unit, a charged particle generation unit, so that the charged particle generation unit generates a charged particle wave at a preset frequency.

2. The method according to claim 1, wherein controlling, by the waveform control unit, the charged particle generation unit comprises the following steps:
reading, by the charged particle generation unit, a charged particle waveform pre-stored in the charged particle generation unit according to a user requirement;
carrying out amplification treatment on the charged particle waveform;
converting the charged particle waveform; and
generating the charged particle wave according to the charged particle waveform and transmitting the generated charged particle wave to the waveform control unit to enable the waveform control unit to regulate the charged particle wave.

3. The method according to claim 1, wherein controlling, by the waveform control unit, the charged particle generation unit comprises the following step:
transmitting, by the waveform control unit, a specific regulating and controlling wave to the charged particle generation unit according to the preset frequency to enable the charged particle generation unit to regulate and control and process particles so as to form the charged particle wave with a specific frequency.

4. An apparatus for realizing an air charged particle wave with a required frequency, comprising:
a charged particle generation unit, wherein the charged particle generation unit is configured to generate charged particles; and
a waveform control unit, wherein the waveform control unit is configured to regulate and control the density and movement direction of the charged particles generated by the charged particle generation unit, such that the charged particles form a charged particle wave moving in a predetermined waveform.

5. The apparatus according to claim 4, wherein the charged particle generation unit comprises:
a storage module configured to pre-store a set charged particle wave by the charged particle generation unit;
an amplification module configured to regulate and control and amplify the charged particle waveform by the charged particle generation unit;
a digital-to-analogue conversion module configured to convert the charged particle waveform by the charged particle generation unit; and
an execution module configured to generate charged particles by the charged particle generation unit according to the particle waveform.

6. The apparatus according to claim 4, wherein the waveform control unit is a fan, configured to regulate and control, and change an output amount of the charged particles by regulating an air speed of the fan.

7. The apparatus according to claim 4, wherein the waveform control unit is an air channel, and the air channel is provided with a closing mechanism, configured to change an output amount of the charged particles by regulating a dimension of the air channel.
